(19) United States
(12) Patent Application Publication
(10) Pub. No.: US 2010/0008898 A1
(43) Pub. Date: Jan. 14, 2010

(54) CHYMOTRYPSIN FROM LUCILIA SERICATA LARVAE AND ITS USE FOR THE TREATMENT OF WOUNDS

(76) Inventors: David Idris Pritchard, Breachfield (GB); Adele J. Horobin, Nottingham (GB); Alan Brown, Nottingham (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/303,010

(22) PCT Filed: May 31, 2007
(86) PCT No.: PCT/GB2007/050307

§ 371 Date: Sep. 23, 2009

Related U.S. Application Data
(60) Provisional application No. 60/837,982, filed on Aug. 15, 2006.

(30) Foreign Application Priority Data
May 31, 2006 (GB) 0610697.5

(51) Int. Cl. A61K 38/48; C12N 9/76

(52) U.S. Cl. 424/94.64; 435/213

(57) ABSTRACT

The use of larval enzymes, particularly a chymotrypsin, is described herein. The enzymes are usable in the treatment of wounds for debridement and for cell regeneration.

This invention relates to larval enzymes. More particularly, the present invention relates to one or more larval enzyme obtainable from Lucilia sericata, which enzymes are useful in tissue regeneration and wound healing.

Observations of the beneficial effects of larval infestation of some wounds were made many years ago. However, further investigations into the mechanisms involved in this process were not conducted until the prevalence of antibiotic resistant strains of infection became problematical in medicine, particularly in the area of wound healing.

Previous studies have looked at the role of larval enzymes in wound debridement (the removal of necrotic, infected or foreign material from a wound), in infection control (the prevention or treatment of infection), as additives or adjuvants to conventional antibiotics and, more recently, in the promotion of tissue regeneration to allow the new tissue in the wound to grow and to close the wound (healing). The larval enzymes used may be obtained by washing the larvae to remove any excretory or secretory products found on the surface of the larva, from the haemolymph or from homogenate of the whole larva, with or without washing of the larvae beforehand. Previous studies have also investigated the effect of rearing the larvae in sterile or non-sterile conditions, of using homogenate, haemolymph or excretion/secretion, and of the age of the larvae (newly hatched or first or second instar). All of these factors have been found to influence the nature or the activity of the products obtained from the larva.

The present inventors have recently focused on the wound healing properties of the larval products, especially enzymes, that is the constituents of the larval product which promote closure of the wound by promoting tissue growth. Surprisingly, the present inventors have found that one or more chymotrypsin enzymes found in the excretion/secretion (surface washing) of the larvae of Lucilia sericata play a crucial role in tissue formation: such enzymes are usually associated with tissue breakdown.

Accordingly, the present invention provides an isolated chymotrypsin derived from insect larvae or an analogue or a synthetic version thereof.

Advantageously, the chymotrypsin of the present invention has been shown to exert dual and conflicting properties in that it is useful not only in the debridement of a wound (the removal of necrotic, infected or foreign material) which may be an expected property of a protease, but also in promoting fibroblast adhesion (i.e. tissue growth) which is a wholly unexpected property for a protease enzyme. The chymotrypsin of the present invention has a selective activity; it removes or degrades some tissue, such as wound eschar, but does not remove or degrade all tissue, such as healthy or new tissue. It may be reasonably expected that the or each protease would degrade or remove tissue regardless of type, such as occurs in blowfly strike caused by Lucilia cuprina where necrotic and healthy tissues are degraded. The dual properties of debridement and promotion of cell growth in the same enzyme is unusual and is surprising to one of skill in the art as it would be expected that these properties are conflicting.

The term “eschar” as used herein is intended to define any dead tissue that is cast off from the surface of the skin including that after a burn injury, gangrene, ulcers (especially pressure ulcers), infections (especially fungal infections), late exposure to anthrax or to any other necrotic tissue.

The insect is preferably the greenbottle fly Lucilia sericata.

The chymotrypsin of the present invention is preferably obtained or obtainable from the excretion/secretion of the insect larvae. However it is possible that the same components may be present in the haemolymph or in homogenate and could be obtained from these sources. The chymotrypsin may, for example, be obtained by washing the larvae and collecting the washing medium. The present inventors have previously found that the excretion/secretion (ES) of insect larvae comprises both constitutively-expressed and inducible components including enzymes, hormones and the like. For this reason it is preferred that the growth conditions of the larvae are kept constant. In the most preferred embodiment of the present invention, the excretion/secretions from newly hatched larvae grown in sterile conditions are collected and used.

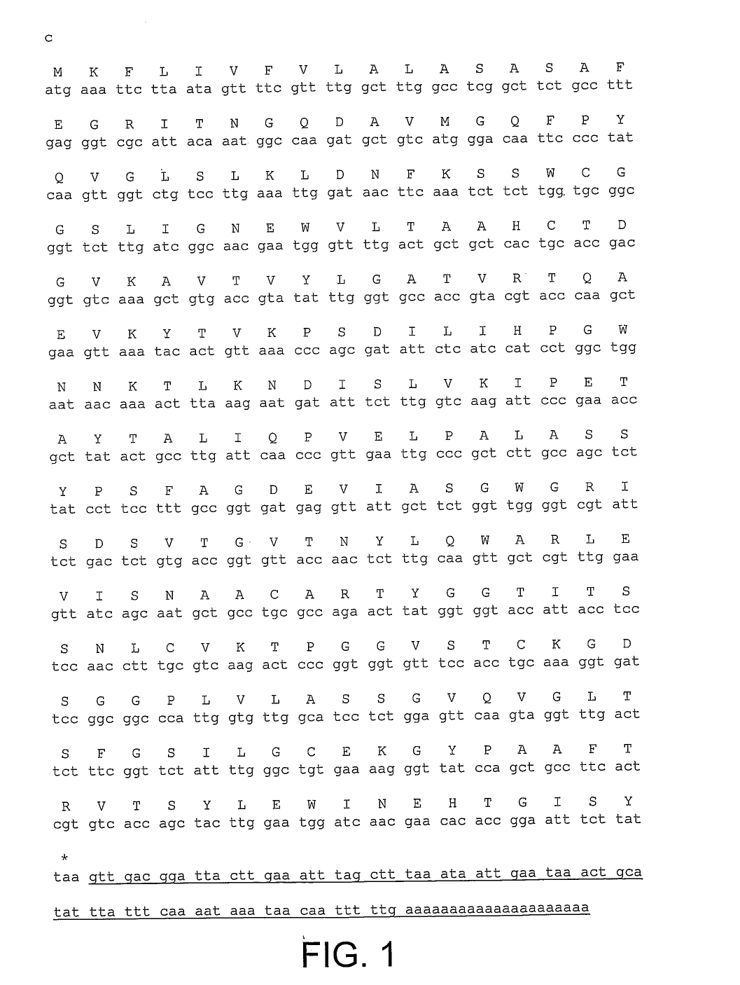

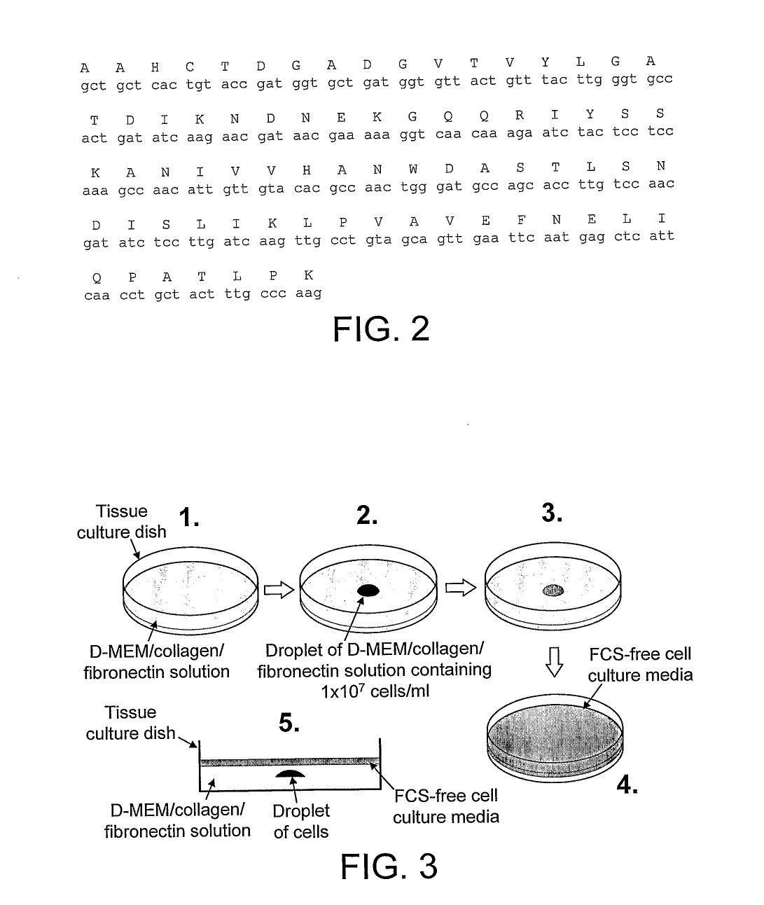

It is preferred that the chymotrypsin has the sequence shown in FIG. 1, FIG. 2 or in FIG. 13 or biologically active peptide fragments thereof which fragments retain the activity of the natural or whole chymotrypsin, the active sites being identified in FIGS. 1, 2 and 13. Preferably, the fragments have high homology to the active sites identified in FIGS. 1, 2 and 13 or to the DNA encoding the peptide fragment of the chymotrypsin, for example the homology to the active site should be at least 90%, preferably above 95% and more preferably 99% homology. Ideally the fragments should have identity to the active site of the peptide or to the DNA encoding the peptide fragment of the chymotrypsin. The relevant DNA sequences are shown in FIGS. 1, 2 and 12.

The chymotrypsin of these sequences has been shown to have tissue regeneration properties and debridement activity but does not degrade or digest healthy or living tissue which is especially surprising given its high homology to a chymotrypsin derived from Lucilia cuprina, the causative agent of blowfly strike (a tissue degenerative process), which has well-known tissue degradation properties even against healthy or living tissue. This homology is also shown in FIGS. 1 and 2.

The present inventors have found that the tissue remodelling and regeneration properties of the ES is lost or inhibited following incubation with the serine proteinase inhibitor phenyl methanesulphonyl fluoride (PMSF) which inhibits trypsin-like and chymotrypsin-like serine proteinases, but was not lost/inhibited following incubation with 4-(amidinophenyl) methane sulphonyl fluoride (APMSF) which only inhibits trypsin-like serine proteinases.

Hence, it was concluded that the important or even essential component of the ES responsible for the extra cellular matrix remodelling or tissue remodelling and tissue regeneration activity was a chymotrypsin or chymotrypsin-like enzyme. This was confirmed by sequencing, as will be described below, where the sequence of the chymotrypsin having the dual properties was identified. As the Examples show, the tissue regeneration properties of the ES are lost when this chymotrypsin is removed, and this chymotrypsin debrides wound eschar, hence the same chymotrypsin enzyme has the dual activity of tissue regeneration and debridement.

The chymotrypsin of the present invention is usable in the treatment of wounds to promote the healing thereof not only by debriding or improving debridement, but also by promoting fibroblast migration, matrix remodelling and the modification of fibroblast morphology. The fact that these opposite effects (proteolysis and protein generation) are present in the same enzyme are surprising to the person skilled in the art and are not predictable from the prior art, especially given the high homology to the chymotrypsin form Lucilia sericata which causes blow-fly strike, a disease which destroys both necrotic and living tissues.

Accordingly, the present invention provides the use of chymotrypsin in a composition for the healing of wounds.

The present invention also provides the use of chymotrypsin in the preparation of a medicament for the healing of wounds, and a method of treating a wound using chymotrypsin. Preferably, the chymotrypsin used in the composition or the medicament has the sequence of FIG. 1, FIG. 2 or FIG. 13, or is encoded by the DNA sequence of FIG. 1, 2 or 12.

Alternatively and active fragment of the chymotrypsin may be used in such a composition or medicament. The chymotrypsin, or the fragment thereof, may be natural or synthetic. It is preferably obtained from the larvae of Lucilia sericata. The wounds of the present invention are defined below and may or may not be infected. Preferably, the wound is a chronic wound, and may have been resistant to conventional therapy.

The wound may be treated by the promotion of fibroblast migration, by the promotion of matrix remodelling or by the modification of fibroblast morphology. Preferably, the wound is debrided by the chymotrypsin of the present invention.

In a further aspect, the present invention also provides a dressing for a wound, the dressing containing the chymotrypsin described as forming part of the present invention. Accordingly, the present invention also encompasses a method of treating a wound, the method comprising the step of applying the abovedescribed dressing to a wound in need of treatment.

The present inventors found that incubating the ES with Soybean Trypsin Inhibitor (STI) removes the ability of the ES to enhance fibroblast migration and its ability to degrade extra-cellular matrix proteins. Similarly, the proteolytic (debridement) activity was altered when the chymotryptic activity was removed, although, as will be shown below, some tryptic activity remained. The protein/s removed by the STI was sequenced. The sequences were found to be chymotrypsin or chymotrypsin like sequences as shown in FIGS. 1, 2 and 13 and confirm that this enzyme and its homologues are responsible for the activity hereindescribed.

Accordingly, the present invention also provides a chymotrypsin having the sequence shown in FIG. 1, FIG. 2 or in FIG. 13. The present invention also provides a DNA sequence encoding the chymotrypsin shown in FIGS. 1, 2 and 13 and a DNA sequence (FIG. 12) showing the L. sericata chymotrypsinogen sequence in pBac-3 vector multiple cloning site DNA sequence. Fragments of the DNA which encode the active sites of the chymotrypsin (see FIG. 13) also comprise part of the present invention.

The term “wound” as used herein is intended to define any damage to the skin, epidermis or connective tissue whether by injury or by disease and as such is taken to include, but not to be limited to, cuts, punctures, surgical incisions, ulcers, pressure sores, burns including burns caused by heat, freezing, chemicals, electricity and radiation, dermal abrasion or assault, osteomyelitis and orthopaedic wounds. The wound may be infected. Additionally, the wound may be chronic or acute. Chronic wounds originate from various conditions and include diabetic foot ulcers, venous leg ulcers, infected surgical wounds, orthopaedic wounds, osteomyelitis and pressure sores.

The chymotrypsin of the present invention may be natural or may be a synthetic version of the natural enzyme such as that shown in FIG. 13. Synthetic versions of the enzyme may be made in conventional manner from the sequence given, for example using a recombinant vector expression system or by use of any known peptide synthesis method or apparatus as shown in the examples which follow. Active fragments of the enzyme, that is fragments of the enzyme which maintain the function of the enzyme, especially those comprising the active sites identified in FIG. 13, are also considered to be part of this invention as are the DNA fragments which encode them.

The chymotrypsin of the invention may be used as an extract which may be crude or purified or it may be incorporated into a pharmaceutical composition comprising conventional additives such as solvents, diluents, buffers, vehicles, stabilisers, humectants, excipients, binders, adjuvants, preservatives, anti-caking agents, acidifying agents, gelling agents, emulsifiers, colourings, fragrances, and the like, especially those used in topical formulations. In a preferred embodiment the chymotrypsin will be applied topically, but it is not intended that oral or other parenteral modes of administration are to be excluded from the scope of this invention. Hence, in the preferred mode of administration, topical administration, the chymotrypsin of the invention may be in an irrigation solution, suspension, a wash, cream, lotion, gel, unguent, ointment, salve, powder or solid or fluid delivery vehicle. Alternatively, the chymotrypsin can be incorporated or encapsulated into a suitable material capable of delivering the chymotrypsin into a wound in a slow release or controlled release manner. An example of such a suitable material is poly (lactide-co-glycolide) or PLGA particles which may be formulated to release peptides in a controlled release manner. Most preferably, the chymotrypsin may be incorporated into a dressing to be applied to the wound. Examples of such dressings include staged or layered dressings incorporating slow-release hydrocolloid particles containing the chymotrypsin, or sponges containing the chymotrypsin optionally overlayered by conventional dressings, see for example those described in Smith et al 2006. Hydrocolloid dressings of the type currently in use, for example those sold under the trade name “Granuflex” may be modified to release the chymotrypsin into the wound.

The chymotrypsin of the present invention may be crude or it may be purified using conventional protein purification methods. The chymotrypsin may be protected against aminopeptidase or other enzyme activity, for example by the amidation at COOH, substitution using a non-coded anomalous amino acid and/or CO—NH amide bond replacement by an isotere. Moreover, the chymotrypsin especially a synthesised or other nascent chymotrypsin may be hydroxylated, glycosylated, sulphated, phosphorylated, or otherwise secondary or tertiary processed, especially where such secondary or tertiary processing confers stability, or improved solubility or other desirable properties to the enzyme. It is especially preferred that a synthetic version of the enzyme is secondary or tertiary processed to arrive at a conformation approximating to that of natural enzyme unless to do so would reduce the activity of the enzyme.

Additionally, the present inventors have found that the excretion/secretion products of insect larvae, especially those of Lucilia sericata, can be used in tissue culture or tissue matrix modelling to maintain the cells. That is, the excretion/secretion products of Lucilia sericata can be used instead of serum, such as calf serum, to maintain the viability of the cells. This is of particular importance where such cells are to be transplanted or grafted onto or into a wound as it removes a potential source of infection, such as the prions associated with Creutzfeldt-Jakob disease (CJD), or of disease transference by removing the need for the use of serum.

Hence the present invention also provides a medium for use in the maintenance of viable cells, the medium comprising the excretion/secretion products of insect larvae. Preferably, the larvae are those of Lucilia sericata.

Typically, Lucilia sericata larvae, or green bottle fly maggots, are applied to chronic wounds where conventional treatments have failed. Clinical observations provide evidence that maggots remove necrotic tissue (debridement), promote disinfection and accelerate granulation tissue formation (Sherman et al, 2000; Wollina et al, 2002). In order to elucidate the mechanisms behind these effects, the present inventors have investigated the enzymatic activity present within maggot secretions and/or excretions (ES) (so called because the substances that maggots continually exude may be of secretory and/or excretory origin) that a separate study has shown are released into the wound (Schmidtchen et al, 2003). Evidence of serine proteinase, metalloproteinase and aspartyl proteinase activities was found (Chambers et al, 2003). In addition, the serine proteinase activity present within ES was shown to degrade a variety of common extracellular matrix (ECM) components (Chambers et al, 2003).

The present inventors have also examined the effect of maggot ES upon interactions between human dermal fibroblasts and extracellular matrix (ECM) components (Horobin et al., 2003, 2005) as these play a crucial role in tissue formation (Eckes et al, 2000). Through binding with cell membrane receptors (Giancotti and Ruoslahti, 1999), the ECM provides a scaffold for contact guidance, controlling fibroblast adhesion and directing cell migration (Clark, 1996; Greiling and Clark, 1997). Proteases derived from many sources, including fibroblasts, modulate such interactions. This may be via direct activation of cell surface receptors, influencing fibroblast proliferation (Abe et al, 2000; Dery and Bunnett, 1999) and angiogenesis (Blair et al, 1997) or by indirect methods in which proteolytic breakdown products of ECM components, most notably fibronectin, induce fibroblast migration and chemotaxis (Greiling and Clark, 1997; Livant et al, 2000), re-epithelialisation (Gianelli et al, 1997) and tissue re-modelling (Gould et al, 1997; Werb et al, 1980). Our results have shown that fibroblast adhesion to fibronectin- and collagen-coated surfaces is reduced in the presence of ES (Horobin et al, 2003). More recently, using a two-dimensional in vitro assay, we have shown that fibroblast migration across fibronectin is accelerated by serine proteinases present within ES (Horobin et al, 2005) i.e. the present inventors have associated such enzymatic activities with enhanced fibroblast migration across planar surfaces. However, evidence suggests that cells behave differently within two dimensions, such as those described, than within their familiar three-dimensional in vivo environment (Abbott, 2003; Cukierman et al, 2001; Friedl and Bröcker, 2000). For example, upon planar surfaces, cells present a flattened lamellar appearance. In contrast, cells observed in situ adopt stellate shapes and protrude dendritic-like networks of extensions. They also exhibit different attachments to the surrounding matrix, termed 3D matrix adhesions (Cukierman et al, 2001). In two dimensions, migration across a surface is predominantly a function of adhesion and de-adhesion events because resistance to the advancing cell body above the planar surface is lacking. Within three dimensions, however, matrix barriers force the cells to adapt their morphology, making them either change shape and/or enzymatically degrade ECM components in order to facilitate locomotion. Hence, the present inventors developed three-dimensional in vitro assays in which to observe fibroblast migration and morphology in response to ES.

Thus, the inventors directed their research towards the instigation of a three-dimensional in vitro assay in which to observe fibroblast migration and morphology in response to ES. The establishment of such a model, which more closely represents the microenvironment in which cells are present in vivo, provides for a much better understanding of the importance of interactions between the ECM, resident cells and ES in the wound healing process. It also provides a basis for developing systems in which viable dermal and epidermal cells, held within a supportive, hydrated, biodegradable and bioactive tissue-like matrix, are delivered to an open wound to facilitate healing. Assays were developed containing isolated populations of primary human foreskin fibroblasts (HFF) embedded within gels composed of collagen and fibronectin, both at concentrations deemed optimal for migration (Greiling and Clark, 1997; Friedl and Bröcker, 2000). Collagen gels have been widely used for in vivo-like cell culture and are considered to represent a fair reproduction of the biophysical architecture of the dermis (Friedl and Bröcker, 2000). In addition, cells embedded within collagen gels have been shown to adopt dendritic-like networks of extensions that share some similarity to the in situ-like morphology (Cukierman et al, 2001). Fibronectin was included as this molecule plays a prominent role in directing cell migration into the wound space (Greiling and Clark, 1997). Results demonstrated ES to accelerate fibroblast migration through the gel in a dose-dependent manner. This may have been facilitated by an enhancement of matrix re-modelling and induction of a more well spread cellular morphology. As will be shown in the examples which follow, in comparison with the relevant controls, ES concentrations of 1 and 5 μg/ml significantly increased both the number of migrating cells and the distances they had traveled away from the cell droplet. These concentrations of ES also induced well spread cellular morphologies and, at low population densities, 5 μg/ml ES promoted matrix fibril alignment between cells. In contrast, 10 μg/ml ES, the highest concentration tested, inhibited cell migration but did alter cellular morphology. ES at a concentration of 0.1 μg/ml exerted little effect over the incubation period examined.

From these observations, it may be concluded that ES promoted matrix reorganisation and the exertion of cellular tractional forces. Within the migration assays, the exertion of traction is indicated by the contraction of cell droplet size following detachment of the gel containing 5 μg/ml ES and during liquefaction of the gel exposed to 10 μg/ml ES. Where cells were observed at lower seeding densities, the presence of tractional forces is indicated by the appearance of straight, aligned matrix fibrils held taut between cells, presumably organised in this way by the exertion of opposing tractional forces. It is also indicated by the well-spread cellular morphologies. As Harris and colleagues (1981) observed of fibroblasts embedded within collagen gels, fibroblast traction ‘ . . . is distinct from simple contraction like that of a muscle . . . ’ because ‘ . . . the cells elongate instead of shorten as they compress and stretch the collagen around them’.

That ES enhanced matrix re-modelling by fibroblasts, and in so doing effected the promotion of migration, is perhaps clarified by a model of gel compaction proposed by Bellows et al (1981). In comparing the abilities of different cell types to contract and organise collagen gels, these researchers identified a series of sequential stages of gel compaction, as shown in Table 1. From our observations, it is clear that ES initiated the first three stages of gel compaction, promoting cell attachment and spreading and precipitating the alignment of matrix (most likely collagen) fibres. In so doing, the fourth stage of cell migration was arrived at. The connection between matrix re-modelling and cell migration is further strengthened by the work of Sawhney and Howard (2002) who found that collagen ‘strap’ (patterns of aligned collagen fibrils) formation between clusters of cells embedded within collagen gels precipitates the advancement of cells and directs cell migration towards neighbouring cell clusters (contact guidance).

TABLE I
Sequential stages of gel compaction,
as proposed by Bellows et al, 1981

| Stage | Description |
|---|---|
| 1 | Attachment of cells to collagen |
| 2 | Cellular spreading within the collagen fibre matrix |
| 3 | Organisation and alignment of collagen fibres by cellular processes |
| 4 | Cell migration |
| 5 | Establishment of intercellular contacts |
| 6 | Development of this arrangement into a 3-dimensional, tissue-like, |
| | honeycomb network. |

From these comparisons, it is clear that cells in the presence of ES have displayed similar characteristics of morphology and matrix reorganisation as have been observed of cells embedded within collagen gels by other researchers. Other research did not include maggot-derived products so it has to be asked why such behaviour was not observed of cells within our controls where ES was absent. The answer may be associated with serum, as comparative research involved assays containing serum, whereas here serum was excluded. Evidence for the promotion of cell-matrix interactions by serum is provided by Tomasek et al (1992) who found that removing serum just prior to the release of tethered fibroblast-populated collagen gels inhibited the extent of subsequent gel contraction by the resident cells. Addition of serum to the gels once they had been released caused an immediate contraction. Other researchers have also observed dependency on serum for matrix reorganisation (Steinberg et al, 1980; Guidry and Grinnell, 1985). Platelet-derived growth factor (PDGF), transforming growth factor beta (TGFβ) and the bioactive lipid mediator lysophosphatidic acid (LPA) are found in serum and have all been implicated in stimulating matrix reorganisation (Montesano and Orci, 1988; Grinnell et al, 1999; Roy et al, 1999; Toews et al, 2002; Kondo et al, 2004).

It is possible, therefore, that maggot ES contains active components that are also found in serum. Another possibility is that the serine proteinases present within ES, which we have shown previously to accelerate fibroblast migration (Horobin et al, 2005), may contribute through cleaving membrane-bound protease-activated receptors (PARs). This family of G-protein-coupled receptors are activated following their enzymatic cleavage by serine proteinases such as thrombin and trypsin-like enzymes. Such action exposes N-terminal tethered ligands on the receptors, which then bind and activate the cleaved receptors. Activated PARs couple to signalling cascades that affect cell shape, secretion, integrin activation, metabolic responses, transcriptional responses and cell motility (Cottrell et al, 2002). Previous work has shown that thrombin promotes the generation of isometric tension within embryonic chick fibroblasts in collagen gels through proteinase activation of the PAR (Kolodney and Wysolmerski, 1992; Pilcher et al, 1994; Chang et al., 2001). It is interesting to note that the serine proteinases present within ES are active against the thrombin and plasmin substrate Tosyl-Gly-Pro-Arg-AMC (Chambers et al., 2003), which means that ES may exert similar effects upon fibroblasts as does thrombin. Any plasmin-like activity may also be pertinent, as plasmin has been shown to activate zymogen pre-cursors of various matrix metalloproteinases (MMPs) secreted by cells, thus contributing to localised matrix reorganisation (Mignatti et al, 1996).

Maggot ES has also been shown to be active against peptides labile to urokinase-like activity. Urokinase, a serine proteinase otherwise known as urokinase plasminogen activator (uPA), converts plasminogen to its active form plasmin. It also binds with plasminogen activator receptors (uPARs), which studies have shown to be expressed by fibroblasts (Mignatti et al, 1996; Ellis et al, 1993; Behrendt et al, 1993). Once bound, uPARs localise to focal adhesion sites and modulate integrin-mediated function (Wang et al, 1995; Chapman and Wei, 2001; Porter and Hogg, 1998), thus providing a mechanism for inducing a more motile cell phenotype. Indeed, uPAR has been reported to have a signalling role in cell migration, adhesion and chemotaxis (Odekon et al, 1992; Waltz et al, 1993; Gyetko et al, 1994). It is therefore reasonable to speculate that urokinase-like activity within ES may have also contributed to alterations in cell behaviour.

Other actions of ES that may have promoted fibroblast-matrix interactions may be related to its ability to degrade the gel matrix components. This is indicated by the observation that the higher the concentration of ES present, the more rapidly the gel changed in its appearance, becoming more translucent and, at the very highest ES concentration, liquid-like. This is also indicated by previous work in which we demonstrated serine proteinases within ES to degrade collagen and fibronectin (Chambers et al., 2003). Degradation of the gels would have resulted in a relaxation of mechanical tension as the contacts between matrix fibrils and the tissue culture dish surface would have been eroded and the fibril network broken down. A number of studies by researchers who developed the tensional culture force monitor provide evidence that fibroblasts embedded within collagen gels are capable of sensing and reacting to changes in mechanical tension. They found that fibroblasts maintain an active tensional homeostasis, reacting to modify the endogenous matrix tension in the opposite direction to externally applied loads (Brown et al, 1998; Eastwood et al, 1994). Hence, an increase in the applied load elicits a decrease in cell-mediated contraction and vice versa. Perhaps contributing to this response is the finding that mechanical tension influences levels of MMP production, thus allowing the cells to react by releasing proteinases to remodel the surrounding matrix (Lambert et al, 2001). Thus, relaxation of mechanical tension caused by proteolytic degradation of the gel may have stimulated the cells to contract and remodel the matrix around them and to adopt a more migratory phenotype. Another consequence may have been the release of bioactive peptides from degraded collagen and fibronectin, which have been shown to influence fibroblast adhesion and migration (Greiling and Clark, 1997; Livant et al., 2000).

As fibroblasts are capable of detecting and responding to changes in mechanical tension, it is clear from our observations that however ES stimulated fibroblasts to remodel the matrix, the enhancement of intercellular communication may have resulted. This is because each cell would have been able to detect local differences in the mechanical tension of the matrix due to opposing tractional forces from other cells causing matrix fibril alignment. Such an enhanced awareness of neighbouring cells, even over comparatively long distances, may have resulted in the improved co-ordination of action between cells, contributing to enhanced migration. Indeed, Sheetz, Felsenfeld and Gabraith (1998) propose a similar mechanism of coordinated cell migration, where the cells direct their movement according to the orientation and rigidity of the ECM protein fibres (Sheetz et al, 1998).

Despite the benefits of ES in enhancing migration and matrix remodelling, it is important that the activity of proteinases present within ES is not excessive as their actions cause a global breakdown of the matrix. That sufficient fibril structure needs to remain in order to facilitate contact guidance and translocation is illustrated by the inventors' assay that contained the highest ES concentration of 10 μg/ml. Here, ES not only inhibited migration but also rapidly degraded the gel into a viscous, liquid state. Clearly then, an optimal concentration of ES exists for promoting cellular activities that may contribute to wound healing.

In summary, our results found that, firstly, ES promoted fibroblast migration. Secondly, in the absence of serum, ES was necessary to help cells maintain and extend well-spread morphologies. Thirdly, again in the absence of serum, ES promoted ECM reorganisation, particularly between cells. Fourthly, ES imposed these effects either by direct proteolytic modification of the ECM and/or by direct alteration of fibroblast phenotype by interaction with cellular receptors, thus supplanting the need for serum. We have the technical ability to isolate the active entities from ES (Chambers et al., 2003; Horobin et al., 2003, 2005) and will therefore proceed to identify the activities and mechanisms involved.

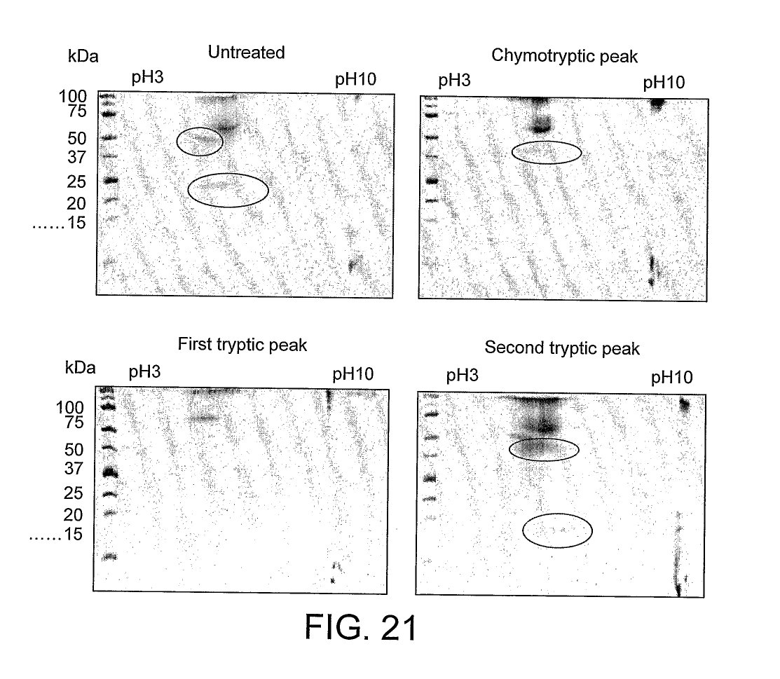

As the data in Example II will show, the chymotrypsin extracted from larval ES has debridement activity in that it lyses proteins in wound eschar (see FIG. 21). It is found to be a chymotrypsin by inhibitor studies and by sequencing.

Examples of the invention will now be described, with reference to and as illustrated by the appended drawings of which

FIGS. 1 and 2 show the full (1) and a partial (2) sequence listing showing the L. sericata gene sequence, with protein translation and the homology to L. cuprina chymotrypsinogen where the DNA codon is in lower case, the protein translation is in upper case; underlined upper case indicate amino acid residues unique to L. sericata sequence when compared with L. cuprina sequence of closest homology; the bold text indicates active site residues; italicised text indicates signalling peptide; arrow indicates possible cleavage site indicating cleavable signalling peptide; asterisk represents stop codon; underlined lower case indicates untranslated region (UTR); bold underlined lower case indicates polyadenylation tail, and the underlined italic text indicates the polyadenylation signal.

FIG. 3 is an illustration of how three-dimensional in vitro assays were assembled. 1. Dulbecco's Modified Eagle's Medium (D-MEM) containing 1.5 mg/ml collagen and 30 μg/ml fibronectin poured into 58 mm tissue culture dish and gelled at 37° C. in a thin, even layer. 2. Droplet of D-MEM/collagen/fibronectin containing 1×10⁷ HFF cells/ml placed on top of the first gel layer and gelled at 37° C. 3. Second layer of D-MEM/collagen/fibronectin solution poured over the top of the cell droplet and gelled at 37° C. 4. FCS-free cell culture medium then poured on top of the gel. 5. Fully assembled assay shown in cross-section, illustrating how all the cells within the droplet are completely surrounded by matrix gel and therefore must migrate in three dimensions.

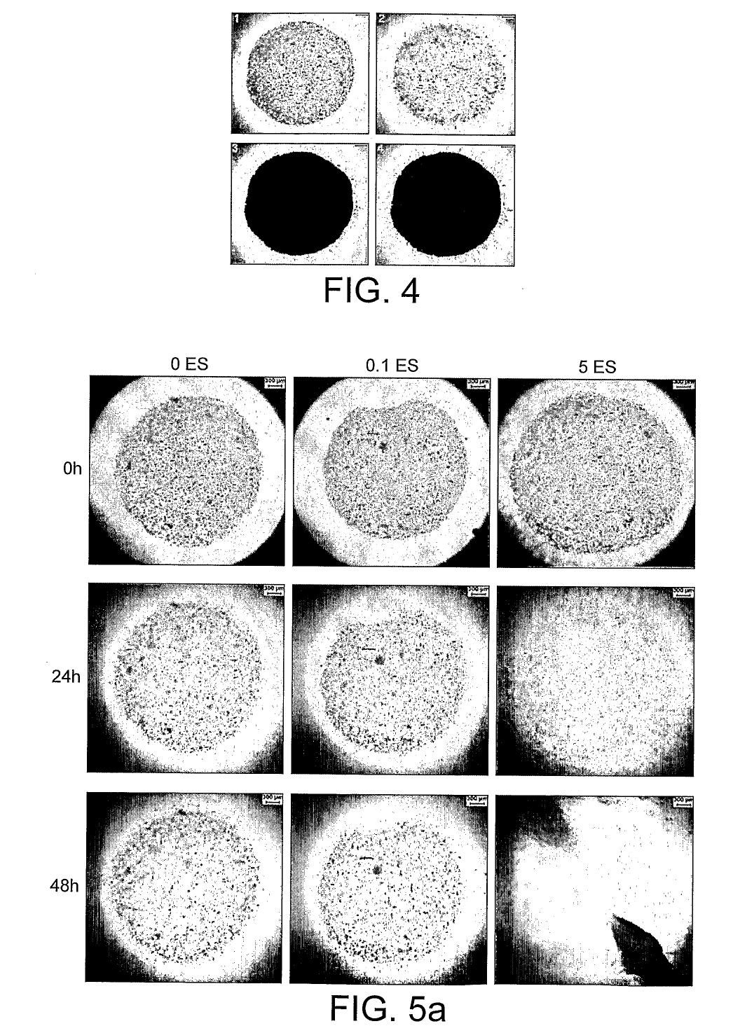

FIG. 4 is an illustration of how fibroblast migration from 2 μl gel droplets within three-dimensional in vitro assays was quantified from phase contrast microscopic images. 1. Fibroblast-seeded droplet immediately after assay assembly (0 h incubation). 2. The same droplet after 24 h incubation. 3. Fibroblast-seeded droplet at 0 h incubation, coloured black for contrast, superimposed upon image from 24 h incubation. 4. Only those cells that had migrated from the droplet over the 24 h period are left showing, thus allowing them to be counted. The distance each cell had traveled was calculated by measuring the lengths of vectors, drawn from the leading edge of each cell, to the perimeter of the superimposed 0 h image. The perimeter distance of the fibroblast-seeded droplet at 0 h incubation was also measured by drawing around the superimposed 0 h image. Micron bars represent 300 μm.

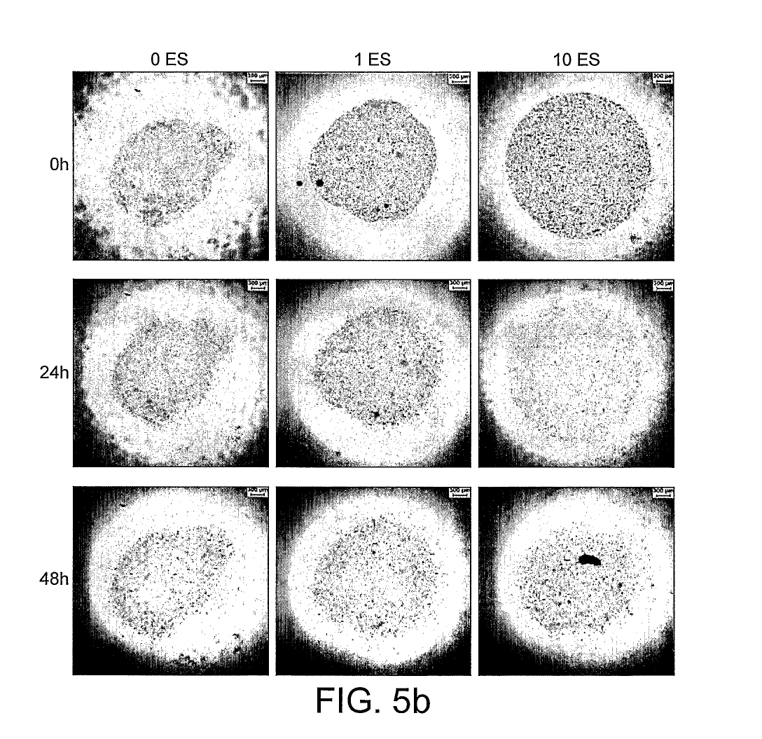

FIG. 5 shows representative phase contrast images of 2 μl fibroblast-seeded gel droplets within three-dimensional in vitro assays immediately following assay assembly (0 h) or after 24 h or 48 h incubation in the a. absence of ES (0 ES) or in the presence of 0.1 μg/ml ES (0.1 ES) or 5 μg/ml ES (5 ES), or b. absence of ES (0 ES) or in the presence of 1 μg/ml ES (1 ES) or 10 μg/ml ES (10 ES). In all cases, micron bars represent 300 μm.

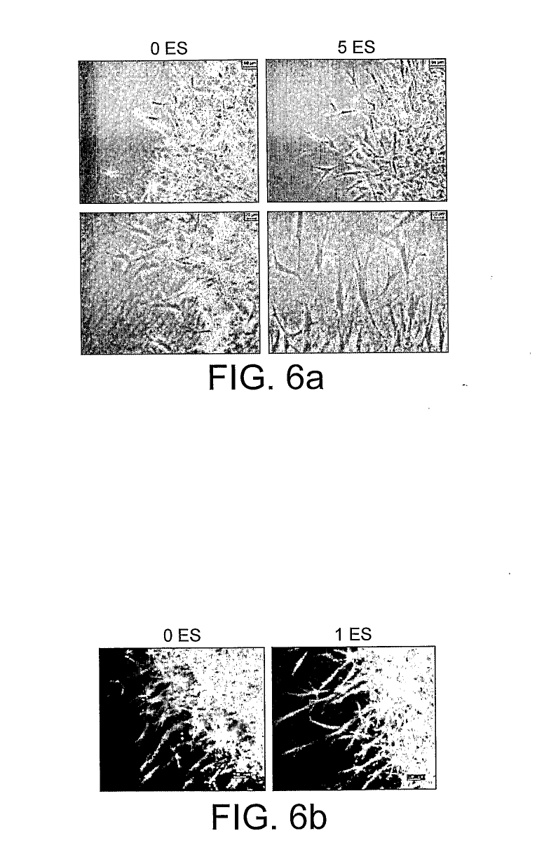

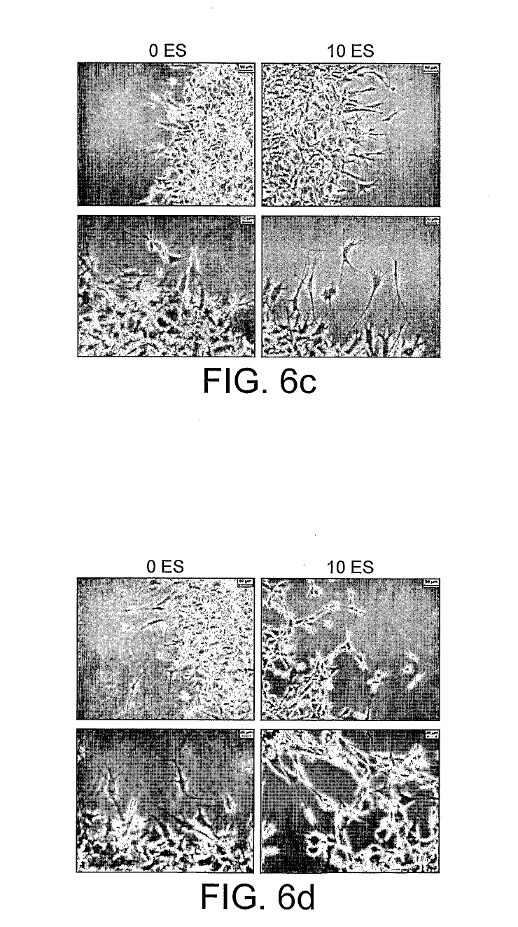

FIG. 6 shows representative images of the edges of 2 μl fibroblast-seeded gel droplets within three-dimensional in vitro assays, highlighting differences in cell morphology. Images are phase contrast, unless otherwise stated. Comparison between a. cells in the control, where ES was absent (0 ES) and cells exposed to 5 μg/ml ES (5 ES) following 24 h incubation; b. cells in the control (0 ES) and cells exposed to 1 μg/ml ES (1 ES) following 48 h incubation—confocal maximum intensity projection of z series optical sections, cells stained with FITC-phalloidin (actin) and propidium iodide (nuclear); cells in the control (0 ES) and cells exposed to 10 μg/ml ES (10 ES) following 24 h incubation (c.) or 48 h incubation (d.) Micron bars in a., c., d. represent 50 μm (upper) or 20 μm (lower) and in b. 50 μm.

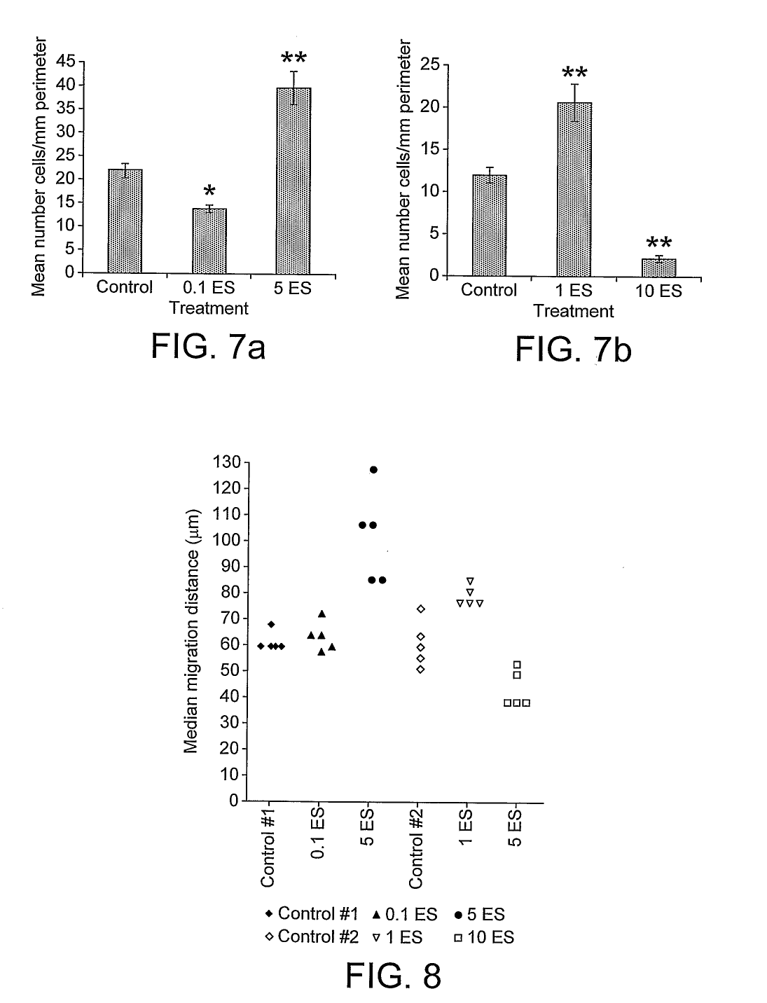

FIG. 7 shows fibroblast migration from 2 μl cell-seeded gel droplets within three-dimensional in vitro assays over 24 h. Results expressed as mean number of migrating cells per mm perimeter of droplet±SEM (n=5). a. Migration in the absence of ES (control) or in the presence of 0.1 μg/ml ES (0.1 ES) or 5 μg/ml ES (5 ES). b. Migration in the absence of ES (control) or in the presence of 1 μg/ml ES (1 ES) or 10 μg/ml ES (10 ES).

FIG. 8 shows median distance traveled by fibroblasts migrating from each 2 μl cell-seeded gel droplet within three-dimensional in vitro assays. Values from five replicate droplets shown. Solid shapes (experiment 1) represent distance traveled in the absence of ES (control #1) or in the presence of 0.1 μg/ml ES or 5 μg/ml ES. Open shapes (experiment 2) represent distance traveled in the absence of ES (control #2) or in the presence of 1 μg/ml ES or 10 μg/ml ES.

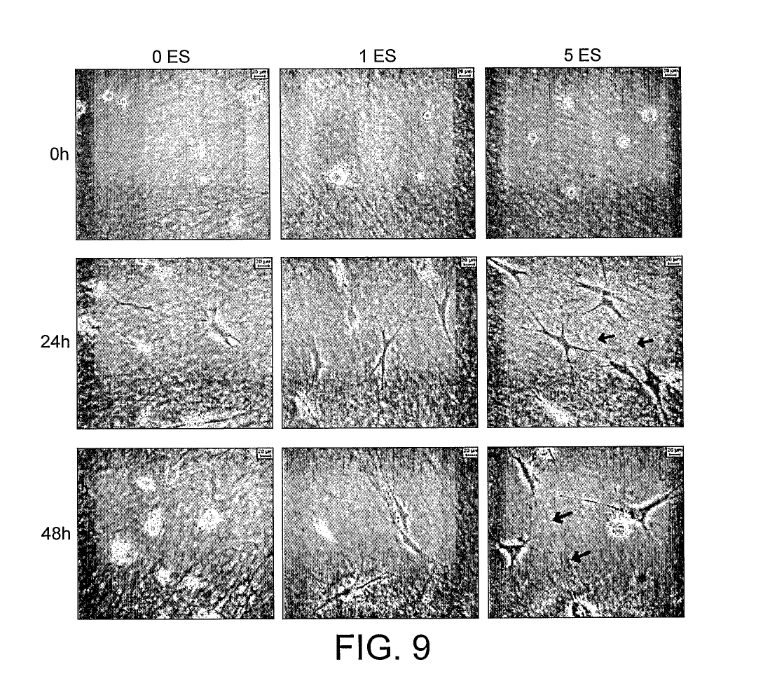

FIG. 9 shows representative phase contrast images showing fibroblasts within 20 μl gel droplets, at a seeding density of 3×10⁵ cells/ml, immediately following assay assembly (0 h incubation) or after 24 h or 48 h incubation. Appearance of cells in the absence of ES (0 ES) (control) or in the presence of 1 μg/ml ES (1 ES) or 5 μg/ml ES (5 ES). Black arrows indicate, where aligned, strand-like connective fibrils between cells have become visible. In all cases, micron bars represent 20 μm.

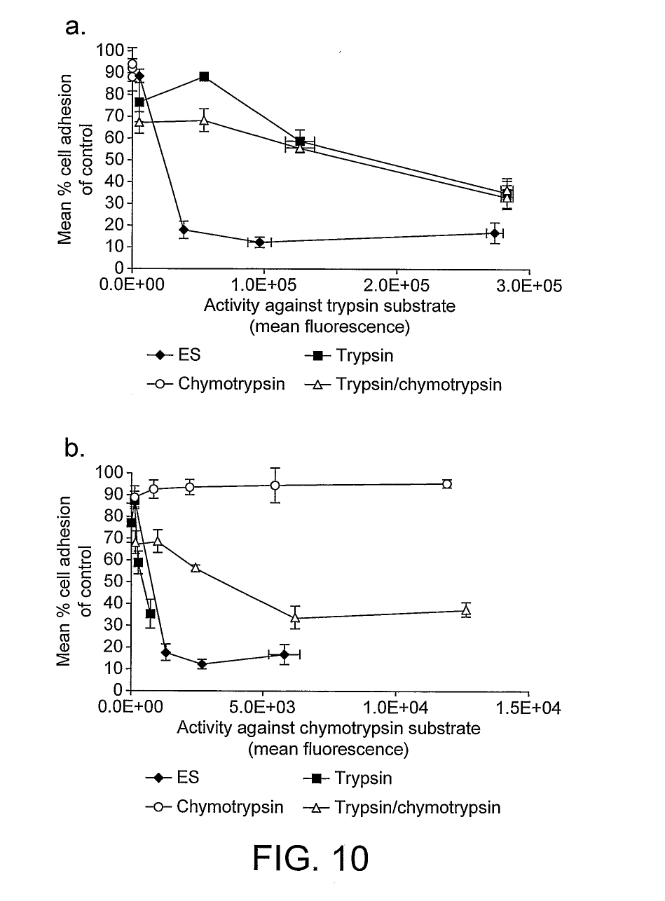

FIG. 10 is two graphs showing the effect of ES, trypsin, chymotrypsin and trypsin/chymotrypsin mix upon fibroblast cell adhesion to a FN-coated surface, following 24 h incubation, in relation to each enzyme concentration's proteolytic activity. Such activity was assessed by monitoring the release of 7-amino-4-methylcoumarin (AMC) from synthetic peptide substrates for a. trypsin or b. chymotrypsin. Cell adhesion was measured using a firefly luciferase-based luminescent adenosine triphosphate (ATP) assay. Results were expressed as mean % cell adhesion of the control±1SD (n=3).

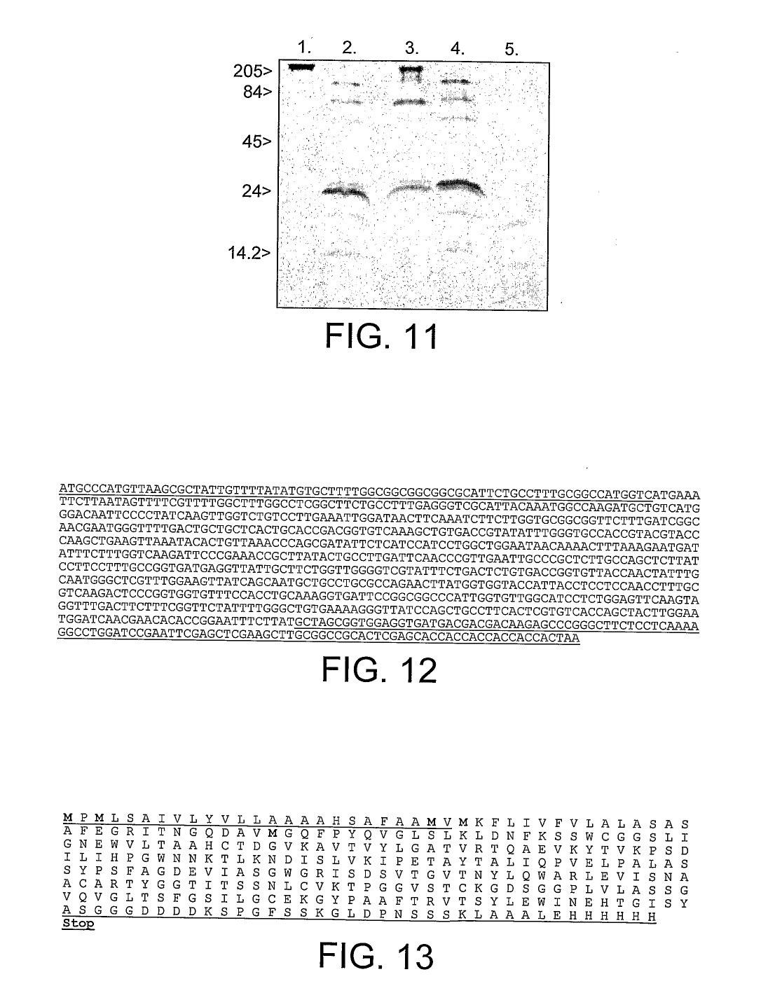

FIG. 11 is a photograph of a gel electrophoresis of fibronectin following incubation at 37° C. for 24 h. Values indicate molecular weight standards (kDa). 1. FN alone. 2. FN+try/chy. 3. FN+chy. 4. FN+try. 5. FN+ES. Each enzyme concentration displayed the same level of activity against the relevant trypsin or chymotrypsin substrate. Try=0.32 μg/ml trypsin. Chy=0.02 μg/ml chymotrypsin. ES=1 μg/ml.

FIG. 12 is a DNA sequence listing showing the L. sericata chymotrypsinogen sequence in pBac-3 vector multiple cloning site DNA sequence where the pbac-3 vector sequence is underlined.

FIG. 13 is the predicted protein sequence of the DNA sequence of FIG. 12 where the active site residues are shown in bold.

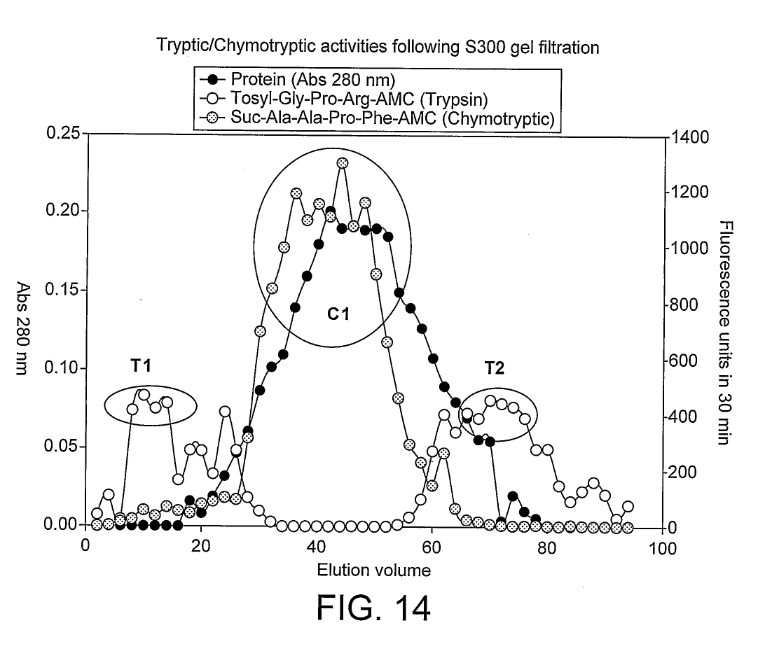

FIG. 14 is a graph showing tryptic/chymotryptic activities following S300 gel filtration.

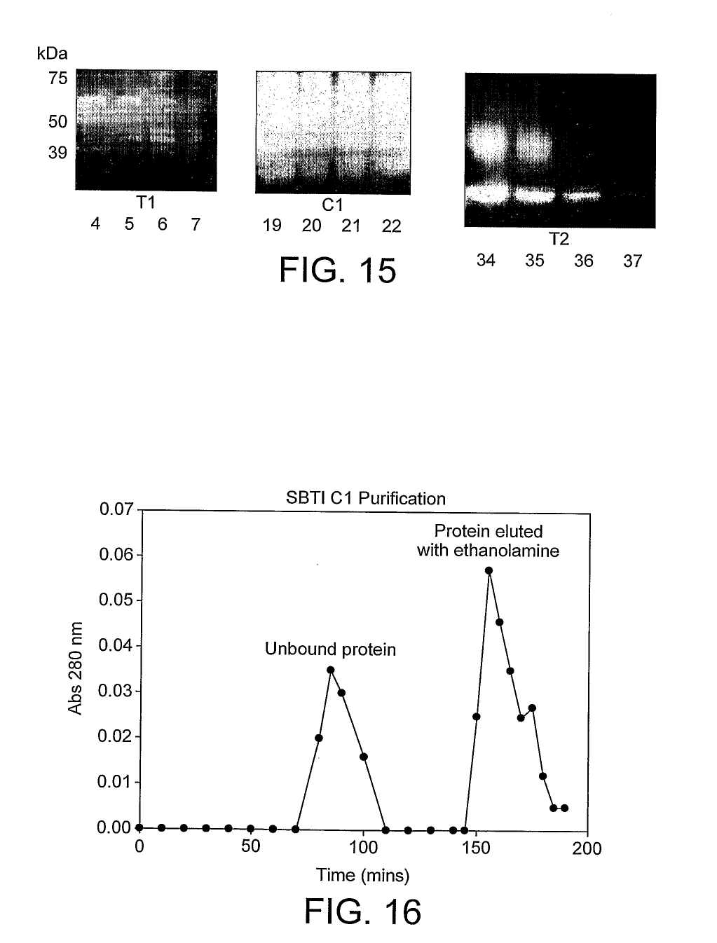

FIG. 15 is a series of photos showing typical proteolytic profiles for each peak of activity as determined by gelatin SDS-PAGE substrate gel analysis.

FIG. 16 is a graph showing the protein profile of soybean trypsin inhibitor agarose affinity chromatography following the application of the C1 pool.

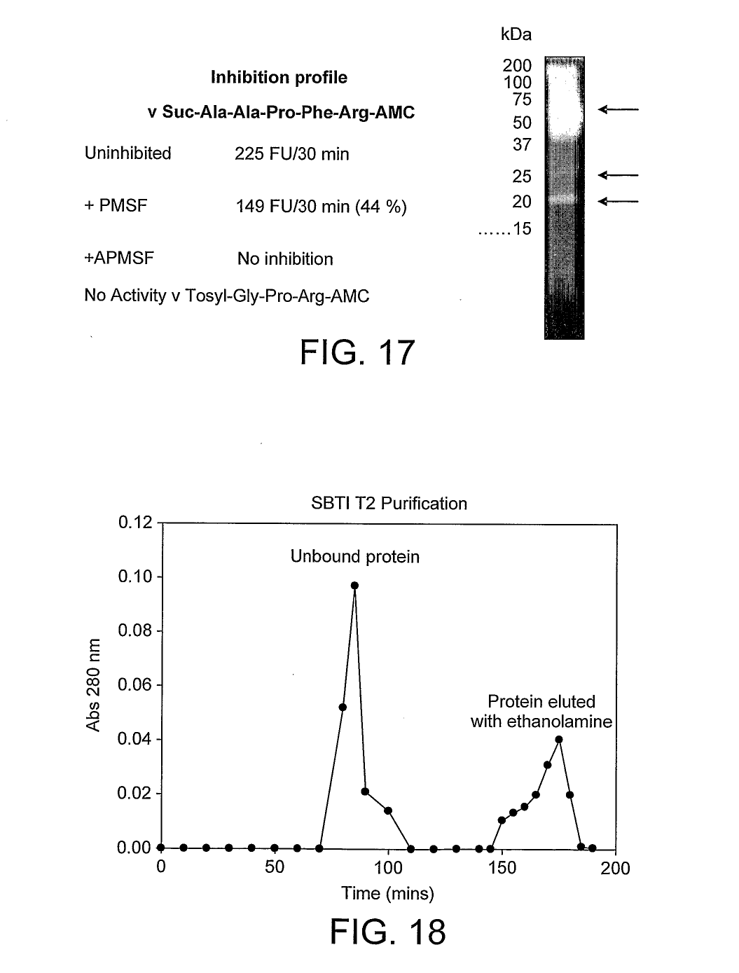

FIG. 17 is a photo of a gel showing the proteolytic activity and inhibitor characterization of protein bound to a soybean trypsin inhibitor agarose following the application of the C1 pool.

FIG. 18 is a graph showing the protein profile of soybean trypsin inhibitor agarose affinity chromatography following the application of the T2 pool.

FIG. 19 is a photo of a gel showing the proteolytic activity and inhibitor characterization of protein bound to a soybean trypsin inhibitor agarose following the application of the T2 pool.

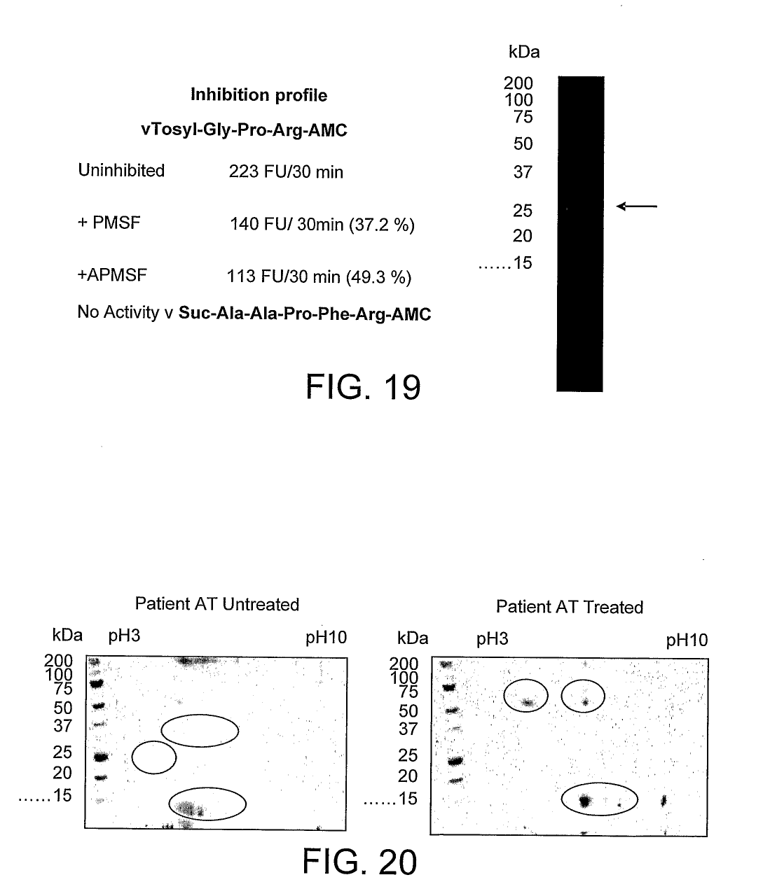

FIG. 20 is a photo showing 2 gels which show the potential changes in protein profile of wound eschar following treatment with L. sericata ES products. Potential areas of change are circled. FIG. 20a shows untreated eschar and FIG. 20b shows eschar treated overnight.

FIG. 21 is a photo showing 4 gels which show the potential changes in protein profile of wound eschar following treatment with L. sericata ES products. Potential areas of change are circled. FIG. 21a shows untreated eschar, 20b treated with chymotrypsin peak (C1), 20c treated with the first tryptic peak (T1) and 20d with the second tryptic peak (T2).

### EXAMPLES

### Example 1

### Tissue Regeneration

### Promotion of Fibroblast Adhesion

Lucilia sericata Larval Excretion/Secretion Collection and Characterisation

Excretions/secretions (ES) were collected from sterile, freshly hatched Lucilia sericata larvae (LarvE™, Zoobiotic Ltd, UK) as previously described (Horobin et al., 2003). Briefly, four hundred live larvae were washed in 1 ml of sterile, phosphate buffered saline (PBS) for 30 minutes at room temperature (RT), to recover ES products. ES protein concentration and proteolytic activity was determined using the Bio-Rad (Hercules, Calif.) protein assay kit and a fluoroscein isothiocyanate (FITC)-casein digest assay (Horobin et al, 2003) respectively. Together, these revealed a protein concentration of 161.74 μg/ml and a specific activity of 6.04×10⁶ relative fluorescence units per mg ES protein.

### Primary Human Foreskin Fibroblast (HFF) Cell Culture

HFF cells (TCS Cellworks®, UK) were monolayer cultured within T75 flasks (Nunc, Life Technologies Ltd, UK), containing Dulbecco's Modified Eagle's Medium (D-MEM) (Gibco™, Invitrogen Ltd, UK), 10% foetal calf serum (FCS) (Sigma®, UK), antibiotic/antimycotic solution (Sigma®) (100 units/ml penicillin, 100 μg/ml streptomycin and 0.25 μg/ml amphotericin B) and 2 mM L-glutamine (Sigma®). Cells were maintained at 37° C. in a humidified atmosphere containing 5% CO₂.

### Three-Dimensional In Vitro Assay—Fibroblast Migration

A stock solution containing D-MEM, antibiotic/antimycotic and L-glutamine at twice the concentrations used for routine cell culture (see above) was made. Following refrigeration, the stock solution was mixed on ice, at a ratio of 1:1, with a cold solution containing 3 mg/ml bovine collagen type I (ICN Biomedicals, Ohio, USA), 60 μg/ml bovine fibronectin (Sigma®) and either larval ES or PBS blank. Final concentrations of 1×D-MEM, 1.5 mg/ml collagen and 30 μg/ml fibronectin were obtained. Final protein concentration of the larval ES was 0.1 μg/ml, 1 μg/ml, 5 μg/ml or 10 μg/ml, as indicated. The assay was assembled as shown in FIG. 3 and as described here: 1000 μl of the D-MEM-collagen/fibronectin mixture, as described above, was poured into a 58 mm tissue culture dish (Nunc, Life Technologies Ltd) and gelled at 37° C. in an even, thin, continuous layer. HFF cells (passage 6) were trypsinised and suspended in D-MEM containing 10% FCS to neutralise the trypsin. They were then pelleted by centrifugation and resuspended in FCS-free D-MEM. Following cell number estimation using a haemocytometer, the cells were again pelleted and resuspended within the D-MEM/collagen/fibronectin gel mix at a density of 1×10⁷ cells/ml. Five droplets, each containing 2 μl of this cell suspension, were then placed on top of the gel layer within the dish and incubated at 37° C. until they had gelled. Another 1000 μl of the D-MEM/collagen/fibronectin mixture was then poured over the top of the cell droplets to completely cover them and left to gel at 37° C. Finally, FCS-free D-MEM containing antibiotic/antimycotic, L-glutamine and either PBS blank or larval ES at the same concentration as in the gel, was added to cover the top gel layer. The assembled assay was then incubated at 37° C. in a humidified 5% CO₂ atmosphere for the time stated. Aseptic conditions were maintained throughout.

Throughout each experiment cells were observed, for a total period of 48 h, through an inverted Leica (UK) DM IRB microscope using phase contrast. Low magnification images, showing the whole area of each cell droplet embedded within each gel, were used to quantify cell migration following 24 h incubation. Here, Microsoft Paint Shop Pro 6 was used to superimpose the image of each cell droplet at 0 h over the same droplet's image after 24 h incubation. These composite images were then analysed, using Leica QUIPS software, as shown in FIG. 4. Firstly, the distance of the perimeter enclosing each cell droplet at 0 h was calculated. This was followed by a count of the number of cells that had migrated across and away from the original cell droplet perimeter over the 24 h period. In order to correct for variable droplet perimeter distances, the number of migrating cells was expressed as cells per mm perimeter of the original cell droplet boundary. The linear distance each cell had migrated away from the perimeter was also measured.

After 48 h incubation, intact gels were fixed in 4% paraformaldehyde (TAAB, Aldermaston, UK) for 20 minutes and then washed three times with PBS containing 1% bovine serum albumin (BSA) (Sigma®). Ice cold permeabilising solution (pH 7.4) containing 20 mM N-(2-hydroxyethyl)piperazine-N′-(2-ethanesulphonic acid); 4-(2-hydroxyethyl)piperazine-1-ethanesulphonic acid (HEPES), 300 mM sucrose, 50 mM NaCl, 3 mM MgCl₂ and 0.5% Triton X-100 (all from Sigma®) was then added and left for 10 minutes. Gels were again washed with 1% BSA. A 0.08% FITC-phalloidin (Sigma®) solution (in ethanol) was diluted 1:100 with 1% BSA and then added to the gels. After 30 minutes, the gels were washed as before. BSA (1%) containing 10 μg/ml propidium iodide (PI) (Sigma®) was added and left for 1 minute before the gels were washed again.

Following FITC-phalloidin/PI staining, gels were carefully blotted to remove excess liquid and mounted with Bio-Rad fluorescence mounting medium and a coverslip. The gels were visualised using a confocal Leica TCS4D system incorporating a Leica DMRBE upright fluorescence microscope. Maximum intensity images of cell droplet edges were taken to observe migrating cell morphology. Z series of optical sections were also taken of the gels and compared with optical sections taken of separate assays that had been fixed and stained immediately following their assembly (0 h incubation). This was done in order to demonstrate that cell migration occurred in all directions from the droplets.

### Statistical Analysis

Values, representing the number of migrating cells per mm perimeter of each cell droplet replicate within each treatment, were transformed to their square roots. These were then subjected to one-way analysis of variance (ANOVA) and Dunnett's Multiple Comparison Tests, using GraphPad Prism™ software. The linear migration distances achieved by cells were compiled under each droplet replicate within each treatment. They were then log transformed to ensure normal distribution and the geometric means collated under the appropriate treatment category. The collated means were analysed using one-way ANOVA and Dunnett's Multiple Comparison Tests, available within GraphPad Prism™. In all cases, equal variance was confirmed and statistical significance was defined as P≦0.05.

### Three-Dimensional In Vitro Assay—Fibroblast Morphology and Matrix Organisation I

In a separate experiment, three-dimensional in vitro assays were assembled, as described above, with minor modifications. Here, one 20 μl droplet, containing a lower cell density of 3×10⁵ cells/ml, was incorporated into each assay. Assays were treated with either 1 μg/ml ES, 5 μg/ml ES or PBS blank (control). Cellular morphology was observed following 0, 24 and 48 hours incubation using phase contrast microscopy. The appearance of the gel matrix was also noted.

### Three-Dimensional In Vitro Assay—Fibroblast Migration

Three-dimensional in vitro assays were assembled in order to examine fibroblast migration. Confocal microscopic images of gels fixed immediately following assay assembly (0 h), or following 48 h incubation, confirmed that, firstly, no cells were observed outside of the cell droplets at 0 h incubation and, secondly, cell migration away from the cell droplets over 48 h had occurred in both horizontal and vertical orientations, confirming the three-dimensional nature of migration (Horobin A J PhD thesis, 2004).

In quantifying cell migration in response to larval ES, two separate experiments were performed. One compared the effects of 0.1 μg/ml ES and 5 μg/ml ES against a control where ES was absent. The other examined the effects of 1 μg/ml ES and 10 μg/ml ES, when compared with another control. In each experiment, cells from the same flask and passage were used.

Images were taken to examine cell migration from each droplet. Visual observations revealed that, at 24 h incubation, migration of cells in the presence of 5 μg/ml ES appeared to be the most extensive (FIG. 5a). Morphologically, these cells also appeared more well spread than those within the control and had projected longer and more numerous extensions into the surrounding gel (FIG. 6a). By 48h incubation, the gel exposed to 5 μg/ml ES was transparent and had become detached from the dish surface. The cell droplets had contracted, pulling the matrix inwards, thus increasing tension within the matrix between droplets. By this time, cells exposed to 1 μg/ml ES appeared to have migrated further than in the respective control (FIG. 5b) and had also adopted a well spread morphology, with long extensions into the surrounding gel (FIG. 6b). In contrast, cell droplets in the presence of 10 μg/ml ES had reduced in size, with some areas of cells contracting into tight, dark masses (FIG. 5b). This may have been related to the now clear, viscous liquid state of the previously gelled solution. When compared with the control, the morphology of cells exposed to 10 μg/ml ES was clearly different. Here, at 24 h incubation, cells exhibited numerous slender extensions, some of which were exceptionally long (FIG. 6c). At 48 h incubation, cells had contracted, becoming rounded, yet they had maintained direct physical contact with others through the projection of long, slender intercellular extensions (FIG. 6d).

Total cell migration from each droplet was quantified using low magnification images that could view each droplet in its entirety. At 24 h incubation, analysis confirmed that, in comparison with the relevant control, 5 μg/ml ES significantly enhanced cell migration, both in terms of the number of cells migrating (P<0.01) (FIG. 7) and the distances traveled (P<0.001) (FIG. 8). ES at 1 μg/ml also significantly enhanced cell migration above its control (P<0.01 for cell number and P<0.05 for distances traveled) (FIGS. 7, 8). In contrast, 10 μg/ml ES significantly inhibited cell migration compared with its control (P<0.01 for cell number and P<0.001 for distances traveled) (FIGS. 7, 8). Exposure to 0.1 μg/ml ES appeared to slightly inhibit the number of cells migrating (P<0.05 against the control) (FIG. 7). However, 0.1 μg/ml ES had no significant impact upon migration distance compared with the control (P>0.05) (FIG. 8), nor did it appear to affect cell morphology (data not shown). It may be noted that the number of cells migrating in the experiment 1 control (FIG. 7a) was higher than in the experiment 2 control (FIG. 7b). Such differences between experiments may be due to slight differences in cell density within cell droplets brought about by an unavoidable level of error when estimating cell numbers using a haemocytometer. However, such a potential source of variation only exists between experiments and not within them (as the same counted lot of cells was used to assemble each assay within each experiment). Hence, it remains valid to compare results within each experiment but not between them.

### Three-Dimensional In Vitro Assay—Fibroblast Morphology and Matrix Organisation II

Three-dimensional in vitro assays containing lower densities of fibroblast cells were assembled in order to examine fibroblast morphology and the organisation of the matrix. Soon after assay assembly, cells in the presence or absence of ES appeared similar (FIG. 9). However, by 24 h incubation cells in the presence of 1 μg/ml ES, and in particular 5 μg/ml ES, had adopted more well spread morphologies, with longer cytoplasmic extensions, than those in the absence of ES. Aligned strand-like connective matrix fibrils between cells were observed where 5 μg/ml ES was present. At 48 h incubation, differences between assays were more pronounced. By this time, cells had become more rounded in the absence of ES (FIG. 9). Meanwhile, cells exposed to either 1 μg/ml ES or 5 μg/ml ES had maintained well spread morphologies. The aligned strand-like connective fibrils observed between cells exposed to 5 μg/ml ES had become more visible and numerous. In addition, the matrix looked clearer and the random meshwork of fibrils less pronounced.

### Comparative Example I

The present inventors undertook a further experiment to compare the effect of the ES chymotrypsin with a commercially available bovine chymotrypsin. The effect of ES upon fibroblast adhesion to fibronectin was therefore compared with commercially available preparations of trypsin and chymotrypsin. This was achieved by seeding fibroblasts upon a fibronectin-coated surface in the presence of various concentrations of ES, commercial trypsin or commercial chymotrypsin. Following incubation periods of up to 48 hours, samples were aspirated of all media and gently washed, thus removing cells that had failed to adhere to the surface. An adenosine triphosphate (ATP) assay was then applied in order to quantify the relative numbers of cells remaining upon the surface according to the concentration of ATP detected. In comparing the effect of ES and trypsin, which contained comparable levels of activity against a trypsin-specific substrate, ES was more effective at reducing fibroblast adhesion to the surface. For example, after 24 hours incubation, 5 μg/ml ES had reduced cell adhesion to 16.6% of the control (FIG. 10a). Commercial trypsin (1.6 μg/ml) which displayed 103.2% of the activity of ES against the trypsin-specific fluorogenic substrate Tosyl-Gly-Pro-Arg-AMC reduced cell adhesion to 34.9% of the control. After 48 hours incubation, cell adhesion in the presence of 5 μg/ml ES had decreased to 9.3% of the control while 1.6 μg/ml commercial trypsin had reduced cell adhesion to 24.7% of the control.

Commercial chymotrypsin (0.1 μg/ml), which displayed 94.1% of the activity of 5 μg/ml ES against a chymotrypsin-specific fluorogenic substrate (Suc-Ala-Ala-Pro-Phe-AMC), demonstrated no ability to modify fibroblast adhesion (FIG. 10b). Even 0.2 μg/ml commercial chymotrypsin, displaying 204.9% of the activity of 5 μg/ml l ES against the chymotrypsin-specific substrate, had no effect upon fibroblast adhesion. Fibroblasts exposed to both commercial trypsin and commercial chymotrypsin simultaneously did not display any modification of adhesion greater than that of cells exposed to a similar concentration of commercial trypsin alone.

These results indicate that the enzymes such as the chymotrypsin present within ES are more effective than commercial trypsin or commercial chymotrypsin alone, or in combination, in modifying fibroblast adhesion. Previous studies have shown that the ES's ability to modify fibroblast adhesion is related to its ability to modify the fibronectin-coated surface via proteolytic degradation (Horobin et al 2003 supra). These results therefore suggest that ESs may be more effective than commercial trypsin or commercial chymotrypsin in degrading extracellular matrix proteins. This is confirmed using gel electrophoresis. Here, samples of larval ES, commercial trypsin, commercial chymotrypsin or commercial trypsin and commercial chymotrypsin combined, were diluted to obtain solutions of similar activity against trypsin-specific or chymotrypsin-specific substrates. Bovine fibronectin (100 μg/ml l) was then exposed, at 37° C., to these solutions for 24 h before being resolved on a 12% polyacrylamide gel. As shown, ES degraded fibronectin more extensively into predominantly smaller fragments when compared with both of the commercial enzymes (FIG. 11). In addition, the proteolytic enzymes present within ES have been shown to enhance fibroblast migration (Horobin et al 2005 supra). This may be associated with alterations in fibroblast adhesion. Within the wound, the acceleration of fibroblast migration may promote granulation tissue growth into the wound space. As such, enzymes derived from ESs may not only prove to be more effective debriding agents than those currently on the market but may also simultaneously enhance the wound healing response.

### Example 2

### Debridement

### Purification of Maggot Chymotrypsin

Sephacryl S300 Chromatography of L. sericata ES Products.

A glass chromatography column (1.5 cm×50 cm) was packed with Sephacryl S-300 HR and equilibrated with PBS (flow rate 0.33 ml/min). The column was calibrated and the void volume determined using broad range gel filtration standards (200-12.5 kDa, Sigma). Approximately 2 ml of L. sericata ES products (0.5 mg) were applied to the column and following the elution of the void volume 50 fractions (2 ml/fraction) were collected and assayed for chymotrypsin and trypsin activity using the fluorescent substrates Suc-Ala-Ala-Pro-Phe-AMC and Tosyl-Gly-Pro-Arg-AMC respectively. See FIGS. 14 and 15 which show chymotrypsin/trypsin activity following S300 gel filtration. Typically 3 peaks of proteolytic activity are observed, two enriched for trypsin (termed T1 and T2) and one for chymotrypsin activity (termed C1). FIG. 15 also illustrates typical proteolytic profiles for each peak of activity as determined by gelatin SDS-PAGE substrate gel analysis.

### Assay of Chymotryptic/Tryptic Activities Using Fluorescent Synthetic Peptide Substrates

Chymotryptic/tryptic activity was assessed by monitoring the release of 7-amino-4-methylcoumarin (AMC) from Suc-Ala-Ala-Pro-Phe-AMC (chymotryptic) and Tosyl-Gly-Pro-Arg-AMC (tryptic). 50 μl of each fraction was incubated with 150 μl of substrate (5 μM) diluted in PBS. Samples were incubated at 37° C. for 30 minutes after which the fluorescence was measured (excitation 365 nm, emission detection 465 nm) using a Dynex MFX microplate fluorimeter. Proteolytic activity is expressed as the number of fluorescence units emitted over 30 minutes following the deduction of a time zero reading.

### Assay of Chymotryptic/Tryptic Activities Using Gelatin Substrate SDS-Page

Substrate SDS-PAGE was carried out using a method described by Kumar & Pritchard (1992). 12% (w/v) SDS-PAGE gels were prepared with the inclusion of 0.1% (w/v) gelatin in the resolving gel. The gel was warmed to 55° C. in order to dissolve the gelatin. 10 μl of each fraction was mixed with an equal volume of non-reducing sample buffer (0.5 M Tris, pH 6.8, 5% SDS (w/v), 20% glycerol (w/v), 0.01% bromophenol blue) and incubated at 37° C. for 30 minutes. The fractions were then applied to individual wells formed in the stacking gel and the sample electrophoresed at a constant current of 20 mA. Following electrophoresis, the gels was washed in 2.5% Triton X-100 for 20 min at room temperature to re-nature the enzymes as described by Lacks & Springhorn (1980). The gels were then washed in water for 20 minutes, and finally incubated overnight at 37° C. in PBS. Proteolytic activity was detected by staining gels with Coomassie brilliant blue R250, and is observed as areas of clear banding against a blue background.

### Purification of Chymotrypsins by Affinity Chromatography on Soybean Trypsin Inhibitor Agarose

The salt concentration of the C1 pool of chymotrypsin activity was adjusted to 0.5 M NaCl and applied to a 3 ml Soybean trypsin inhibitor agarose column equilibrated with PBS, 0.5 M NaCl (flow rate 0.2 ml/min). The column was washed with PBS, 0.5 M NaCl until the absorbance at 280 nm of the buffer passing through the column reached zero. Bound protein was eluted with 0.7 % ethanolamine and 1 ml fractions were collected which were immediately neutralized with 2M Tris.Cl (1:1 v/v), pooled and dialysed overnight against PBS (FIG. 16). The eluted protein was assayed for proteolytic activity using fluorescent substrates and gelatin SDS-PAGE (FIG. 17).

FIG. 17 shows the proteolytic activity and inhibitor characterization of protein bound to a soybean trypsin inhibitor agarose following the application of the C1 pool. Protein eluting from a soybean trypsin inhibitor agarose column cleaved the chymotryptic substrate Suc-Ala-Ala-Pro-Phe-Arg-AMC and was shown to be inhibitable only by PMSF. No activity was seen against the tryptic substrate Tosyl-Gly-Pro-Arg-AMC. When analysed by gelatin substrate SDS-PAGE at least 3 distinct areas of proteolytic activity were observed (arrowed).

### Purification of Trypsin by Affinity Chromatography on Soybean Trypsin Inhibitor Agarose

Similarly, the T2 pool of tryptic fractions was passed down a Soybean trypsin inhibitor column. Fraction eluting from the column were immediately neutralized, pooled and dialysed overnight against PBS (FIG. 19).

FIG. 19 shows the proteolytic activity and inhibitor characterization of protein bound to a soybean trypsin inhibitor agarose following the application of the T2 pool. Protein eluting from a soybean trypsin inhibitor agarose column cleaved the tryptic substrate Tosyl-Gly-Pro-Arg-AMC and was shown to be inhibitable by PMSF and APMSF. No activity was seen against the chymotryptic substrate Suc-Ala-Ala-Pro-Phe-Arg-AMC. When analysed by gelatin substrate SDS-PAGE 1 distinct band of proteolytic activity was observed (arrowed).

### Effects of ES Products on Wound Eschar

Approximately 1 mg of wound eschar was incubated overnight with 10 μg of L. sericata ES products. Following incubation the treated and untreated eschar was dissolved into Biorad iso-electric focusing re-hydration buffer (8M Urea, 2% CHAPS 50 mM DTT, 0.2% Ampholytes). Protein was the further purified using the Biorad 2D protein ‘cleanup’ kit, the resulting pellet being re-solublised in iso-electric focusing re-hydration buffer.

The effect of ES products on wound eschar were then examined by 2D gel analysis. Approximately 100 μg of protein was focused in the first dimension using ‘ReadyStrip’ 7 cm IPG strips pH 3-10 (Biorad) under conditions described by the manufacturer. Second dimension SDS-PAGE was carried out using 10% tricene gels (Schagger and Vonjagow, 1987). Gels were stained with Coomassie blue R250 FIG. 20.

### The Effects of Chymotrypsin/Trypsin Pools on Wound Eschar

Approximately 1 mg of wound eschar was incubated overnight with 100 μl of L. sericata C1 .T1 or T2 pools. Following incubation the treated and untreated eschar was processed as described above.

The effect of the C1 .T1 or T2 pools on wound eschar was then examined by 2D gel analysis. Approximately 100 μg of protein was focused in 2 dimensions as described above. Gels were stained with Coomassie blue R250 FIG. 21.

Cloning a L. sericata Chymotrypsin Gene

The full length open reading frame (ORF) of L. sericata chymotrypsinogen was amplified from a cDNA library by PCR using a forward primer adding a NcoI restriction site (5′-CTGCCATGGTCATGAAATTCTTAATAGTT-3′) and a reverse primer adding a NheI site (5′-GACGCTAGCATAAGAAATTCCGGTGTG-3′). The resulting fragment was cloned into pBac-3 downstream of the polyhedrin promoter and sequenced (FIG. 12) and the amino acid sequence predicted (FIG. 13).

It can therefore be concluded that the ES of Lucilia sericata contain a chymotrypsin enzyme having the amino acid and DNA sequences shown herein and which both debrides a wound and promotes healing of the wound by promotion of fibroblast migration, by promotion of matrix remodelling and by modification of fibroblast morphology.

### REFERENCES

Abbott A: Biology's new dimension. Nature 424: 870-872, 2003
Abe M, Kurosawa M, Ishikawa O, Miyachi Y: Effect of mast cell-derived mediators and mast cell-related neutral proteases on human dermal fibroblast proliferation and type I collagen production. J Allergy Clin Immunol 106 Suppl: 78-84, 2000
Behrendt N, Ploug M, Ronne E, Høyer-Hansen G, Dano K: Cellular receptor for urokinase-type plasminogen activator: protein structure. Methods Enzymol 223: 207-222, 1993
Bellows C G, Melcher A H, Aubin J E: Contraction and organization of collagen gels by cells cultured from periodontal ligament, gingival and bone suggest functional differences between cell types. J Cell Sci 50: 299-314, 1981
Blair R J, Meng H, Marchesse M J, Ren S L, Schwartz L B, Tonnesen M G, Gruber B L: Human mast cells stimulate vascular tube formation. Tryptase is a novel, potent angiogenic factor. J Clin Invest 99 (11): 2691-2700, 1997
Brown R A, Prajapati R, McGrouther D A, Yannas I V, Eastwood M: Tensional homeostasis in dermal fibroblasts: mechanical responses to mechanical loading in three-dimensional substrates. J Cell Physiol 75: 323-332, 1998
Chambers L, Woodrow S, Brown A P et al: Degradation of extracellular matrix components by defined proteases from the green bottle fly larva Lucilia sericata used for chemical debridement of non-healing wounds. Br J Dermatol 148: 14-23, 2003
Chang M C, Chan C P, Wu H L, Chen R S, Lan W H, Chen Y J, Jeng J H: Thrombin-stimulated growth, clustering and collagen lattice contraction of human gingival fibroblasts is associated with its protease activity. J Periodontol 72 (3): 303-313, 2001
Chapman H A, Wei Y: Protease crosstalk with integrins: the urokinase receptor paradigm. Thromb Haemost 86: 124-129, 2001
Clark R A F: Wound repair: overview and general considerations. In: Clark R A F (ed). The molecular and cellular biology of wound repair. Second Edition. New York: Plenum Press, 1996; p 3-50
Cottrell C S, Coelho A M, Bunnett N W: Protease-activated receptors: the role of cell-surface proteolysis in signalling. Essays Biochem 38: 169-183, 2002
Cukierman E, Pankov R, Stevens D R, Yamada K M: Taking cell-matrix adhesions to the third dimension. Science 294: 1708-1712, 2001
Déry O, Bunnett N W: Proteinase-activated receptors: a growing family of heptahelical receptors for thrombin, trypsin and tryptase. Biochem Soc Trans 27: 246-254, 1999
Eastwood M, McGrouther D A, Brown R A: A culture force monitor for measurement of contraction forces generated in human dermal fibroblast cultures: evidence for cell-matrix mechanical signalling. Biochim Biophys Acta 1201: 186-192, 1994
Eckes B. Zigrino P. Kessler D, Holtkøtter O, Shephard P, Mauch C, Krieg T: Fibroblast-matrix interactions in wound healing and fibrosis. Matrix Biol 19: 325-332, 2000
Ellis V, Behrendt N, Dano K: Cellular receptor for urokinase-type plasminogen activator: function in cell-surface proteolysis. Methods Enzymol 223: 223-233, 1993
Friedl P, Bröcker E-B: The biology of cell locomotion within three-dimensional extracellular matrix. Cell Mol Life Sci 57: 41-64, 2000
Giancotti F G, Ruoslahti E: Integrin signalling. Science 285: 1028-1032,
Gianelli G, Falk-Marzillier J, Schiraldi O, Stetler-Stevenson W G, Quaranta V: Induction of cell migration by matrix metalloprotease-2 cleavage of laminin-5. Science 277: 225-228, 1997
Gould L J, Yager D R, Cohen I K, Diegelmann R F: In vitro analysis of foetal fibroblast collagenolytic activity. Wound Repair Regen 5: 151-8, 1997
Grieling D, Clark R A F: Fibronectin provides a conduit for fibroblast transmigration from collagenous stroma into fibrin clot provisional matrix. J Cell Sci 110: 861-70, 1997
Grinnell F, Ho C-H, Lin Y-C, Skuta G: Differences in the regulation of fibroblast contraction of floating versus stressed collagen matrices. J Biol Chem 274(2): 918-923, 1999
Guidry C, Grinnell F: Studies on the mechanism of hydrated collagen gel reorganization by human skin fibroblasts. J Cell Sci, 79: 67-81, 1985
Gyetko M R, Todd III R F, Wilkinson C C, Sitrin R G: The urokinase receptor is required for human monocyte chemotaxis in vitro. J Clin Invest 93: 1380-1387, 1994
Harris A K, Stopak D, Wild P: Fibroblast traction as a mechanism for collagen morphogenesis. Nature 290: 249-251, 1981
Horobin A J, Shakesheff K M, Pritchard D I: Maggots and wound healing: an investigation of the effects of secretions from Lucilia sericata larvae upon the migration of human dermal fibroblasts over a fibronectin-coated surface. Wound Repair Regen 13 (4): 422-433, 2005
Horobin A J, Shakesheff K M, Woodrow S, Robinson C, Pritchard D I: Maggots and wound healing: an investigation of the effects of secretions from Lucilia sericata larvae upon interactions between human dermal fibroblasts and extracellular matrix components. Br J Dermatol 148: 923-933, 2003
Kolodney M S, Wysolmerski R B: Isometric contraction by fibroblasts and endothelial cells in tissue culture—a quantitative study. J Cell Biol 117 (1):
Kondo S, Kagami S, Urushihara M et al: Transforming growth factor-β1 stimulates collagen matrix remodelling through increased adhesive and contractive potential by human renal fibroblasts. Biochim Biophys Acta 1693 (2): 91-100, 2004
Kumar, S. & Pritchard, D. I. (1992) The partial characterisation of proteases present in the Excretory/Secretory products and exsheathing fluid of the infective (L₃) larva of Necator americanus. International Journal for Parasitology, 22, 563-572.
Lacks, S. A. & Springhorn, S. S. (1980) Renaturation of enzymes after polyacrylaminde gel electrophoresis in the presence of sodium dodecyl sulphate. Journal of Biological Chemistry, 255, 7467-7473.
Lambert C A, Colige A C, Munaut C, Lapiere C M, Nusgens B V: Distinct pathways in the over-expression of matrix metalloproteinases in human fibroblasts by relaxation of mechanical tension. Matrix Biol 20: 397-408,
Livant D L, Brabec R K, Kurachi K et al: The PHSRN sequence induces extracellular matrix invasion and accelerates wound healing in obese diabetic mice. J Clin Invest 105: 1537-1545, 2000
Mignatti P, Rifkin D B, Welgus H G, Parks W C: Proteinases and tissue remodelling. In: Clark R A F (ed). The molecular and cellular biology of wound repair. Second Edition. New York: Plenum Press, 1996; p 427-474
Montesano R, Orci L. Transforming growth factor β stimulates collagen-matrix contraction by fibroblasts: Implications for wound healing. Proc Natl Acad Sci USA 85: 4894-4897, 1988
Odekon L E, Sato Y, Rifkin D B: Urokinase-type plasminogen-activator mediates basic fibroblast growth factor-induced bovine endothelial-cell migration independent of its proteolytic activity. J Cell Physiol 150 (2): 258-263, 1992
Pilcher B K, Kim D W, Carney D H, Tomasek J J: Thrombin stimulates fibroblast-mediated collagen lattice contraction by its proteolytically activated receptor. Exp Cell Res 211: 368-373, 1994
Porter J C, Hogg N: Integrins take partners: cross-talk between integrins and other membrane receptors. Trends Cell Biol 8: 390-396, 1998
Roy P, Petroll W M, Cavanagh H D, Jester J V: Exertion of tractional force requires the coordinated up-regulation of cell contractility and adhesion. Cell Motil Cytoskeleton 43: 23-34, 1999
Sawhney R K, Howard J: Slow local movements of collagen fibers by fibroblasts drive the rapid global self-organization of collagen gels. J Cell Biol 157 (6): 1083-1091, 2002
Schagger, H. & Vonjagow, G. (1987) Tricine sodium dodecyl-sulfate polyacrylamide gel electrophoresis for the separation of proteins in the range from 1-kDa to 100-kDa. Analytical Biochemistry, 166(2), 368-379.
Schmidtchen A, Wolff H, Rydengard V, Hansson C: Detection of serine proteases secreted by Lucilia sericata in vitro and during treatment of a chronic leg ulcer. Acta Derm-Ven 83 (4): 310-311, 2003
Sheetz M P, Felsenfeld D P, Gabraith C G: Cell migration: regulation of force on extracellular-matrix-integrin complexes. Trends Cell Biol 8: 51-54, 1998
Sherman R A, Hall M J R, Thomas S: Medicinal maggots: an ancient remedy for some contemporary afflictions. Annu Rev Entomol 45: 55-81, 2000
Smith A G, Powis R A, Pritchard D I, Britland S T: Greenbottle (Lucilia sericata) Larval Secretions Delivered from a Prototype Hydrogel Wound Dressing Accelerate the Closure of Model Wounds. Biotechnol. Prog 22: 1690-1696, 2006
Steinberg B M, Smith K, Colozzo M, Pollack R: Establishment and transformation diminish the ability of fibroblasts to contract a native collagen gel. J Cell Biol 87: 304-308, 1980
Toews M L, Ediger T L, Romberger D J, Rennard S I: Lysophosphatidic acid in airway function and disease. Biochim Biophys Acta 1582 (1-3) Sp. Iss: 240-250, 2002
Tomasek J J, Haaksma C J, Eddy R J, Vaughan M B: Fibroblast contraction occurs on release of tension in attached collagen lattices: dependency on an organized actin cytoskeleton and serum. Anat Rec 232: 359-368, 1992
Waltz D A, Sailor L Z, Chapman H A: Cytokines induce urokinase-dependent adhesion of human myeloid cells. J Cell Invest 91: 1541-1552, 1993
Wang N. Planus E, Pouchelet M, Fredberg J J, Barlovatzmeimon G: Urokinase receptor mediates mechanical force transfer across the cell-surface. Am J Physiol—Cell Physiol 37 (4): C1062-C1066, 1995
Werb Z, Banda M J, Jones P A: Degradation of connective tissue matrices by macrophages I. Proteolysis of elastin, glycoproteins and collagen by proteinases isolated from macrophases. J Exp Med 152: 1340-57, 1980
Wollina U, Liebold K, Schmidt W, Hartmann M, Fassler D: Biosurgery supports granulation and debridement in chronic wounds—clinical data and remittance spectroscopy measurement. Int J Dermatol 41: 635-639, 2002

1. An isolated chymotrypsin derived from insect larvae, a synthetic version thereof, or an active fragment thereof.

2. The chymotrypsin of claim 1, wherein the chymotrypsin has the sequence of FIG. 1 or FIG. 13 or is encoded by the DNA sequence of FIG. 1, 2, or 12.

3. The chymotrypsin of claim 1, wherein chymotrypsin is a fragment of the full-length chymotrypsin, and wherein the fragment has the same activity as the full-length chymotrypsin.

4. The chymotrypsin of claim 1, wherein the chymotrypsin is obtained from the larvae of Lucilia sericata.

5. The chymotrypsin of claim 1, wherein the chymotrypsin is from the excretion/secretion of the larvae of Lucilia sericata.

6. The chymotrypsin of claim 1, wherein the larvae are newly hatched.

7. The chymotrypsin of claim 1, wherein the larvae are first instar.

8. The chymotrypsin of claim 1, wherein the larvae are grown in a sterile environment.

9. A method for treating a wound, comprising administering to the wound a composition comprising a chymotrypsin derived from insect larvae, a synthetic version thereof, or an active fragment thereof.

10. The method of claim 9, wherein the chymotrypsin derived from insect larvae, synthetic version thereof, or active fragment thereof is used to prepare a medicament for the treatment of the wound.

11. The method of claim 9, wherein the chymotrypsin has the sequence of FIG. 1, FIG. 2, or FIG. 13, or is encoded by the DNA sequence of FIG. 1, 2, or 12.

12. The method of claim 9, wherein an active fragment of the chymotrypsin is used.

13. The method of claim 9, wherein the chymotrypsin is synthetic.

14. The method of claim 9, wherein the chymotrypsin is derived from Lucilia sericata.

15. The method of claim 9, wherein the wound is selected from the group consisting of cuts, punctures, surgical incisions, ulcers, pressure sores, burns including burns caused by heat, freezing, chemicals, electricity and radiation, dermal abrasion or assault, osteomyelitis and orthopaedic wounds.

16. The method of claim 9, wherein the wound is infected.

17. The method of claim 9, wherein the wound is chronic.

18. The method of claim 9, wherein the wound is treated by the promotion of fibroblast migration.

19. The method of claim 9, wherein the wound is treated by the promotion of matrix remodelling.

20. The method of claim 9, wherein the wound is treated by the modification of fibroblast morphology.

21. The method of claim 9, wherein the wound is debrided by the chymotrypsin, the synthetic version thereof, or the active fragment thereof.

22. A dressing for a wound, wherein the dressing comprises chymotrypsin derived from insect larvae, a synthetic version thereof, or an active fragment thereof.

23. The chymotrypsin of claim 1, wherein the chymotrypsin is incorporated in a pharmaceutical composition comprising a pharmaceutically acceptable additive.

24. (canceled)

25. The method of claim 9, wherein the chymotrypsin is administered to the wound as an extract, on a dressing, or in a pharmaceutical composition.
